(19) United States
(12) Patent Application Publication
(10) Pub. No.: US 2026/0325404 A1
(43) Pub. Date: Oct. 22, 2026

(54) DRUG TRANSFER SYSTEM CONFIGURED FOR ASEPTIC TRANSFER OF LIQUID PRODUCT

(71) Applicant: Oyster Point Pharma, Inc., Princeton, NJ (US)

(72) Inventors: John K. Lokhnauth, Princeton, NJ (US); Eric C. Ehrnsperger, Princeton, NJ (US); Travis D. Buel, Hickman, NE (US); Jason M. Lepree, Demarest, NJ (US); George J. Donato, Princeton, NJ (US); Jeffrey Alan Nau, Princeton, NJ (US); Peter A. Smith, Cary, NC (US); Joseph Vincent Ranalletta, Greenville, SC (US); Eli B. Nichols, Jacksonville, FL (US)

(21) Appl. No.: 19/103,447

(22) PCT Filed: Aug. 16, 2023
(86) PCT No.: PCT/US2023/030331

§ 371 Date: Feb. 12, 2025

Related U.S. Application Data
(63) Continuation-in-part of application No. 17/889,285, filed on Aug. 16, 2022.
(60) Provisional application No. 63/398,494, filed on Aug. 16, 2022.

(51) Int. Cl. A61J 1/20; A61F 9/00; A61M 15/08

(52) U.S. Cl. CPC A61J 1/20; A61F 9/0008; A61M 15/08; A61J 1/201; A61J 1/2037; A61J 1/2055

(57) ABSTRACT

A drug transfer system may include a housing comprising a vial receptacle, a pump, an adapter arranged in the housing, and a cannula. The adapter may include a vial interface portion and a drug delivery device interface portion, a first channel defined at least partially in the vial interface portion, wherein the first channel is in fluidic communication with the pump and the vial receptacle, and a second channel defined a least partially in the drug delivery device interface portion. The cannula may extend at least partially into the vial receptacle and be in fluidic communication with the second channel. The drug transfer system may furthermore be removably coupleable to a drug delivery device, such that the drug delivery device is in fluidic communication with the second channel.

### TECHNICAL FIELD

This invention relates generally to the field of storage and/or delivery of fluids (e.g., drugs).

### BACKGROUND

In many instances, drugs or other medications are transported and/or stored in a form that is different than its form when administered to a patient, and must be prepared in in a certain manner before it can be administered to the patient. For example, a lyophilized drug may be reconstituted into a liquid, or a frozen drug may be thawed. Such preparation of the drug typically occurs in a standalone vial, and then the prepared drug may be accessed with a syringe or other suitable device.

In some instances, the prepared drug must be administered to a patient in a particular manner using a designated type of drug delivery device (e.g., eyedropper, nasal spray pump). In these instances, the drug is often prepared in a separate vial and must be subsequently transferred to the drug delivery device for administration. However, this transfer process is risky and may pose challenges to a user. For example, if the transfer process is not handled properly, it may lead to contamination and/or other degradation of the prepared drug. Accordingly, special training may thus be required to properly transfer the drug from a vial to a drug delivery device. Untrained patients or other consumers may find it difficult or confusing to properly handle the drug, which may lead to a reduction in drug efficacy, danger to the patient, and/or drug product wastage.

Thus, there is a need for a new and improved system and method for aseptic transfer of a liquid product for drug delivery and/or the like.

### SUMMARY

Generally, in some variations, a drug transfer system may include a housing comprising a vial receptacle, a pump coupled to the housing, and an adapter arranged in the housing. The adapter may include a vial interface portion and a drug delivery device interface portion, where a first channel may be defined at least partially in the vial interface portion and is in fluidic communication with the pump and the vial receptacle, and where a second channel may be defined a least partially in the drug delivery device interface portion. The drug transfer system may, in some variations, further include a cannula in fluidic communication with the second channel and extending at least partially into the vial receptacle.

The drug transfer system may be coupleable (e.g., removably coupleable) to a drug delivery device such that the drug delivery device is in fluidic communication with the second channel.

For example, in some variations, the drug transfer system may be removably coupleable to the drug delivery device via the drug delivery device interface on the adapter. In some variations, the adapter may include a luer-compatible interface removably coupleable to the drug delivery device (e.g., threaded interface or other suitable interface).

While the drug transfer system is coupled to a drug delivery device and a vial is arranged in the vial receptacle (so as to be in fluid communication with the first and second channels of the drug transfer system), actuation of the pump may pressurize the interior of the vial through the first channel, thereby driving drug contents of the vial through the second channel and into the drug delivery device in an aseptic transfer process. The drug delivery device may furthermore be decoupled from the drug transfer system, and subsequently operated to administer the drug contained in the drug delivery device in any suitable manner (e.g., via an eyedropper, nasal spray pump, etc.).

In some variations, the drug transfer system may include other air flow management or processing components in the fluidic paths among the pump, first channel, vial receptacle, and second channel. For example, the drug transfer system may include a valve (e.g., one-way valve, such as an umbrella valve) that is in a fluidic communication path between the pump and the vial receptacle, such that the valve permits airflow from the pump into the vial receptacle and substantially prevents airflow in the reverse direction. As another example, the drug transfer system may include an air filter in the fluidic communication path between the pump and the vial receptacle.

In some variations, drug transfer system may further include a sharp guard slidingly engaged with the cannula. The sharp guard may furthermore include one or more members configured to engage a vial, such as a vial that is inserted in the vial receptacle of the housing, to retain the vial within the vial receptacle.

In some variations, the pump may be configured to pressurize the vial receptacle, such as upon manual (or automated) actuation of the pump. The pump may, for example, include a depressible bulb for actuating the pump, but in other example variations the pump may include an actuator such as a button, trigger, piston, slider crank, and/or the like.

A vial may be directly insertable in the vial receptacle, though in some variations the system may include a carrier that is configured to receive the vial (e.g., in a cavity of the carrier) and is insertable in the vial receptacle. In some variations, once the carrier is inserted in the vial receptacle, the carrier may be secured to retain the carrier in position. For example, the housing and/or the carrier may include a lock configured to retain the carrier in the vial receptacle, such as one or more mating or engageable features. As an illustrative example, the housing may include a spring arm that is configured to engage a surface of the carrier.

Generally, in some variations, a method of aseptically transferring a drug to a drug delivery device may include a drug delivery device to a drug transfer device, where the drug transfer device includes a vial receptacle, a pump, a first channel in fluidic communication with the pump and the vial receptacle, and a second channel in fluidic communication with the drug delivery device and extending at least partially into the vial receptacle. In some variations, the second channel may include or be coupled to a cannula configured to extend into the vial. The method may further include inserting into the vial receptacle a vial comprising the drug to be transferred, allowing the first channel and the second channel to extend into the vial, and actuating the pump to drive fluid into the first channel and into the vial, thereby urging the drug through the second channel and into the drug delivery device. The method may further include decoupling the drug delivery device from the drug transfer device, such as after a desired amount of the drug has been transferred in this manner. While in some variations the vial may be directly inserted in the vial receptacle, in some variations the method may include coupling the vial to a carrier that is insertable in the vial receptacle. The carrier may then be locked in the vial receptacle.

### BRIEF DESCRIPTION OF THE DRAWINGS

FIG. 1 depicts an illustrative schematic of an example variation of a drug transfer system coupled to a drug delivery device.

FIGS. 2A and 2B depict a perspective view and an exploded view, respectively, of an example variation of a drug transfer system.

FIG. 3A depicts a partially assembled view of an example variation of a drug transfer system configured to couple to a drug delivery device. FIGS. 3B and 3C depict longitudinal cross-sectional views of an example variation of a drug transfer system receiving a vial therein. FIG. 3D depicts a longitudinal cross-sectional view of a variation of a drug transfer system coupled to a drug delivery device.

FIG. 4 depicts an illustrative schematic of an example variation of a housing in a drug transfer system.

FIG. 5 depicts an illustrative schematic of an example variation of a vial carrier in a drug transfer system.

FIGS. 6A and 6B depict a perspective view and a longitudinal cross-sectional view, respectively, of an example variation of a vial adapter in a drug transfer system.

FIG. 7A depicts an illustrative schematic of an example variation of a drug transfer system operable with a drug delivery device having an eyedropper dispenser.

FIG. 7B depicts an illustrative schematic of an example variation of a drug transfer system operable with a drug delivery device having a nasal spray pump dispenser.

FIGS. 8A-8E depict a perspective view, a partially assembled view, a translucent view, a longitudinal cross-sectional view, and a detailed longitudinal cross-sectional view, respectively, of an example variation of a drug transfer system with an integrated dispenser.

FIG. 9 depict an example variation of a drug transfer system with an integrated dispenser and protective guard for the eye.

FIGS. 10A-10C depict a perspective view, a partially assembled view, and another perspective view, respectively of an example variation of a drug transfer system coupled to a drug delivery device. FIG. 10D depicts an illustrative schematic of the drug delivery device shown in FIGS. 10A-10C decoupled from the drug transfer system.

FIG. 11A depicts a partially assembled view of an example variation of a drug transfer system in which a drug delivery device connected to the drug transfer system functions as the pump.

FIGS. 11B-11C depict a longitudinal cross-sectional view of the variation of a drug transfer system depicted in FIG. 11A coupled to the drug delivery device and in various stages of use.

FIG. 12A depicts a partially assembled view of an example variation of the drug transfer system shown in FIG. 11A that is configured in an inverted configuration.

FIGS. 12B-12C depict longitudinal cross-sectional views of the variation of a drug transfer system depicted in FIG. 12A coupled to the drug delivery device and in various stages of use.

### DETAILED DESCRIPTION

Non-limiting examples of various aspects and variations of the invention are described herein and illustrated in the accompanying drawings.

Described herein are systems and methods for aseptic transfer of a liquid product (e.g., drug) to a device configured to administer the liquid product in a particular manner. For example, a liquid drug (or other suitable fluid product) may be prepared in a suitable preparation vial (e.g., configured to permit reconstitution of a lyophilized drug, or thawing of a frozen drug such as biologics or small molecules), then transferred to a drug transfer system configured to operate with a drug delivery dispenser. The drug transfer system is simple to operate and includes one or more features to help reduce risk of contamination of the liquid product during transfer, as described herein. Additionally or alternatively, the drug transfer system may reduce manufacturing constraints. For example, if a drug were to be stored and transported within its associated drug delivery device beginning from time of manufacture, a customized process may be required to properly fill the drug delivery device with the drug prior to shipping. It would also be challenging to properly sterilize the entire assembled product with the drug and drug delivery device, since the drug and the drug delivery device have different material characteristics and requirements for sterilization (e.g., a typical sterilization process for a drug dispenser may adversely affect the drug). By facilitating an easy way for users (e.g., clinicians or patients at home) to aseptically transfer a drug from a vial to a drug delivery device, the vial and the drug delivery device may be separately assembled and/or sterilized. In other words, the drug delivery device may be sterilized through conventional and well-vetted processes without the drug, while allowing a vial to be filled with the drug and separately sterilized through conventional and well-vetted processes.

Generally, in some variations, a drug transfer system may include a housing including a vial receptacle, and an adapter arranged in the housing. The adapter may include at least a pump, a first channel in fluidic communication with the pump and the vial receptacle, and a second channel that extends at least partially into the vial receptacle. In some variations, the drug transfer system may include a cannula in fluidic communication with the second channel, and either or both the cannula and the second channel may extend at least partially into the vial receptacle. In some variations, the components of the drug transfer system may be individually and/or collectively sterilized during manufacture or assembly of the drug transfer system (e.g., through gamma radiation sterilization), such that the drug transfer system may provide an aseptic fluid path for liquid drug traveling therein.

The drug transfer system may include or be coupleable to a drug delivery device such that the drug delivery device is in fluidic communication with the second channel. The drug delivery device may, for example, include a luer fitting to permit one-way directional flow into the drug delivery device from the second channel. The drug delivery device may include a suitable dispenser (e.g., eyedropper, nasal spray pump). In some variations, the drug delivery device may include a luer fitting. For example, the drug delivery device may be similar to any of the devices described in U.S. patent application Ser. No. 17/889,285, which is incorporated in its entirety by this reference.

In some embodiments, the drug delivery device may comprise a container, the container including a luer compartment and a fluid compartment, with the luer fitting arranged within the luer compartment and in fluidic communication with the fluid compartment, such as described in at least paragraphs [0005], [0009], and [0025] of U.S. patent application Ser. No. 17/889,285. In a further embodiment, the drug delivery device comprises a dispenser in fluidic communication with the fluid compartment.

In some embodiments, the drug delivery device comprises a luer fitting which may include a luer valve configured to receive fluid from a syringe or other fluid dispensing device. In a further embodiment, the luer fitting may be coupled to the luer compartment via a fluidic seal, such as described in at least paragraphs [0005], [0025], and [0027] of U.S. patent application Ser. No. 17/889,285.

In some embodiments, the drug delivery device may include a cap configured to enclose the luer fitting within the luer compartment. For example, the cap may be configured to couple to the container, such as described in at least paragraphs [0005], [0028], [0033], and [0034] of U.S. patent application Ser. No. 17/889,285.

In some embodiments, the luer compartment and the fluid compartment of the drug delivery device may be arranged in any suitable portions or locations of the container. For example, in some variations the luer compartment and the fluid compartment may be arranged on opposite ends of the container, such as described in at least paragraphs [0006], [0042], and [0044] of U.S. patent application Ser. No. 17/889,285.

In some embodiments, the drug delivery device comprises a container and a dispenser, where the dispenser is coupled to the container via a snap fit or threaded interface between the container and the dispenser, such as described in at least paragraphs [0007] and [0026] of U.S. patent application Ser. No. 17/889,285.

In some embodiments, the drug delivery device comprises a dispenser, such as an eyedropper or a nasal spray pump, such as described in at least paragraphs [0008], [0029], and of U.S. patent application Ser. No. 17/889,285.

A preparation vial including a drug to be transferred (e.g., a reconstituted lyophilized drug, a thawed frozen drug, etc.) may be inserted into the vial receptacle in a manner such that the first channel and the cannula are in fluidic communication with the vial. In some variations, the vial may be coupled to a carrier (e.g., cup, sleeve, or other suitable housing) that is configured to engage with the vial receptacle. The carrier may, for example, engage with the vial receptacle so as to lock the vial in the vial receptacle. Once the vial is in place with the first channel and second channel in fluidic communication with the vial, the pump may be actuated (e.g., manually by a user, or through an automated system) to drive air (or another suitable fluid).

Actuation of the pump may drive air or another suitable fluid through the first channel and into the vial, thereby pressurizing the vial and causing the drug within the vial to be urged into the second channel and/or cannula (if present). The drug may continue to be urged through the second channel and into the drug delivery device. Accordingly, actuation of the pump may result in a transfer of the drug from the vial into the drug delivery device. After a desired amount of drug has been transferred, the drug delivery device may be detached from the drug transfer system, and thereafter used to administer the drug to a patient in accordance with the particular dispenser type in the drug delivery device (e.g., eyedropper, nasal spray pump).

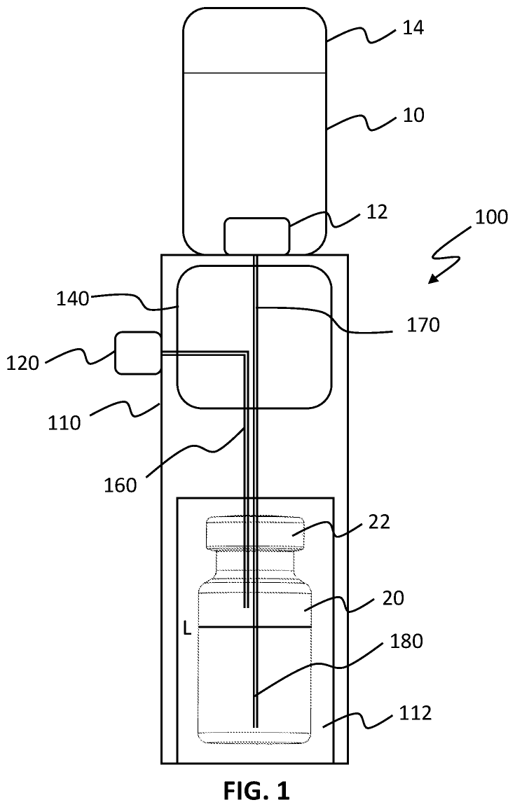

For example, as shown in the schematic of FIG. 1, in some variations, a drug transfer system 100 may include a housing 110 defining a vial receptacle 112, a pump 120 coupled to the housing 110, and a cannula 180 configured to extend at least partially into the vial receptacle. The drug transfer system 100 may also include a first fluidic channel 160 and a second fluidic channel 170. In some variations, the first and second channels 160 and 170 may be defined in an adapter 140. The first channel 160 may be in fluidic communication with the pump 120, and the second channel 170 may be in fluidic communication with the cannula 180. In some variations, the cannula 180 may extend at least partially into the adapter 140 and additionally function as channel 170. Alternatively, in some variations, the channel 170 may extend into the vial receptacle in the absence of a cannular 180.

A vial 20 may be inserted into the vial receptacle 112. The vial 20 may engage with the vial receptacle 112 directly, and/or may be placed within a carrier that engages the vial receptacle 112. A drug delivery device 10 (with dispenser 14) may be coupled to the drug transfer system 100 to receive a fluid from the vial 20. The drug delivery device 10 may, for example, couple to the drug transfer system 100 (e.g., the adapter 140) so as to be in fluidic communication with the second channel 170 and/or the cannula 180. When a vial 20 is inserted into the vial receptacle 112, the first channel 160 and the second channel 170 may enter and extend at least partially into the vial 20. In some variations, the first and second channels may have differing lengths such that they terminate at different heights within the vial 20. Specifically, the second channel 170 may extend further into the vial 20 than the first channel 160. For example, the second channel 170 may extend nearly the entire depth of the vial 20 (e.g., terminating in the liquid drug contents of the vial 20, below level (L)), while the first channel 160 may extend until just past the neck of the vial 20 (e.g., terminating outside the level of the liquid drug contents of the vial 20, above level (L)).

Generally, the drug transfer system 100 may formed through any suitable manufacturing process, such as injection molding, 3D printing, casting, and/or suitable machining processes. Suitable materials for forming any of the components of the drug transfer system 100 include rigid or semi-rigid plastics, though the drug transfer system 100 may include any suitable material.

To operate the drug transfer system 10, a user may actuate the pump 120 to drive air or other suitable fluid through the first channel 160 and into the vial 20. This action may increase the pressure in the vial 20, thereby causing the drug in the vial to be urged into the cannula 180, through the second channel 170, and into the drug delivery device 10 (through the luer fitting 12, if present). In some variations in which the cannula 180 extends fully from the vial 20 to the drug delivery device 10, actuation of the pump 120 may instead cause the drug in the vial 20 to be urged through the cannula 180 and directly into the drug delivery device 10.

Accordingly, actuation of the pump may result in a transfer of the liquid product from the vial 20 into the drug delivery device 10. After a sufficient amount of liquid product has been transferred, the drug delivery device 10 may be detached from the drug transfer system 100. The luer fitting 12 of the drug delivery device 10 may be sealed (e.g., self-sealing septum, covered with a cap component, etc.). Once decoupled from the drug transfer system 100, the drug delivery 10 may be used separately to administer the drug to a patient in accordance with the particular dispenser in the drug delivery device (e.g., eyedropper, nasal spray pump).

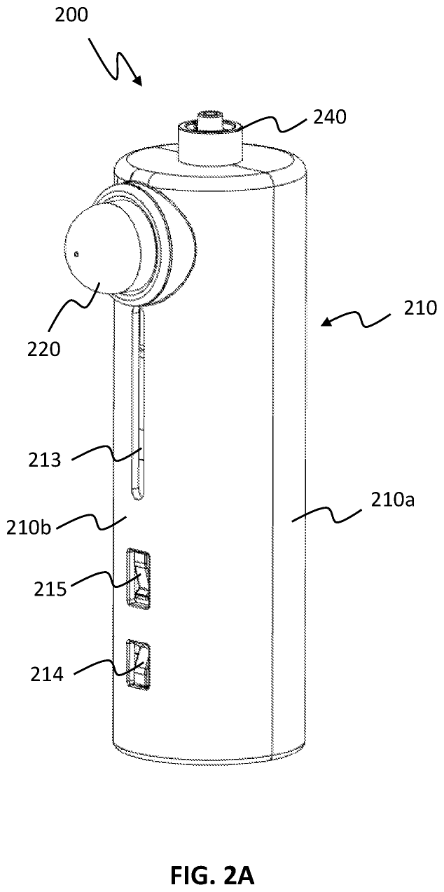

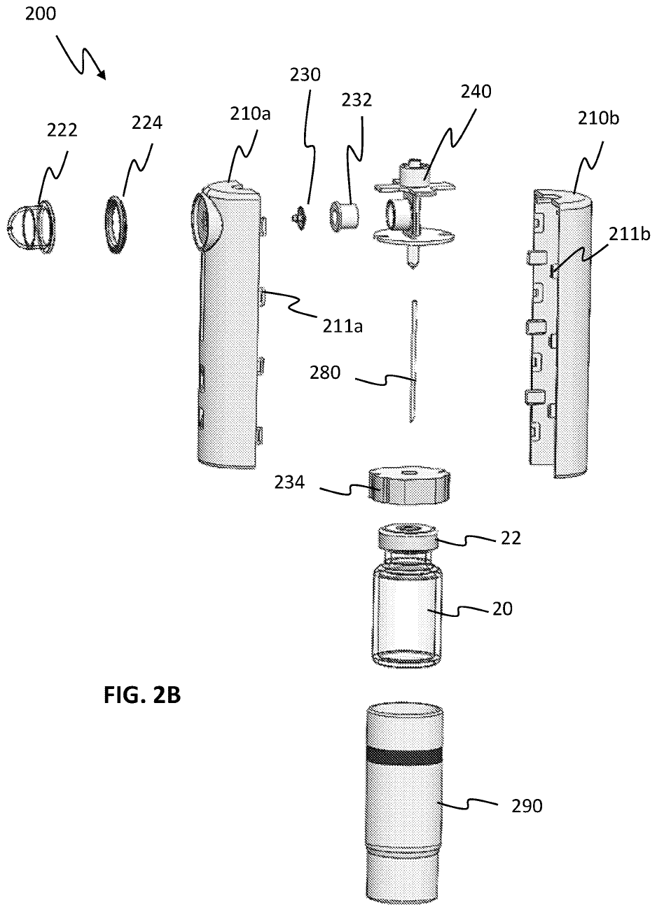

FIGS. 2A and 2B depict another example variation of a drug transfer system 200. As shown in FIG. 2A, the drug transfer system 200 may include a housing 210 and a pump 220 coupled to the housing. The housing 210 is shown as generally cylindrical; however, the housing 210 may have any suitable shape (e.g., prismatic with any suitable polygonal cross-section, tapered, hourglass, etc.). A vial receptacle may be defined in the housing 210. For example, in some variations the housing 210 may include a single unitary piece with a cavity or recess that functions as a vial receptacle to receive a vial. Alternatively, in some variations the housing 210 may include multiple housing portions that may couple together to form the vial receptacle. For example, as shown in FIGS. 2A and 2B, the housing 210 may include two housing portions 210a and 210b, each of which has a generally semi-circular cross-section. The housing portions 210a and 210b may include housing shells that couple together to form an enclosed space, at least a portion of which may function as the vial receptacle. The housing portions 210a and 210b may couple together in any suitable manner, such as via a suitable mechanical interfit (e.g., snap fit) or fasteners. For example, as shown in FIG. 2B, a first housing portion 210a may include one or more slots 211a that engage with one or more tabs 211b on a second housing portion 210b to secure the housing portions together. Although FIGS. 2A and 2B depict a housing 210 formed from two generally mirrored halves, it should be understood that the housing 210 may alternatively be formed from any suitable number of pieces (three, four, etc.).

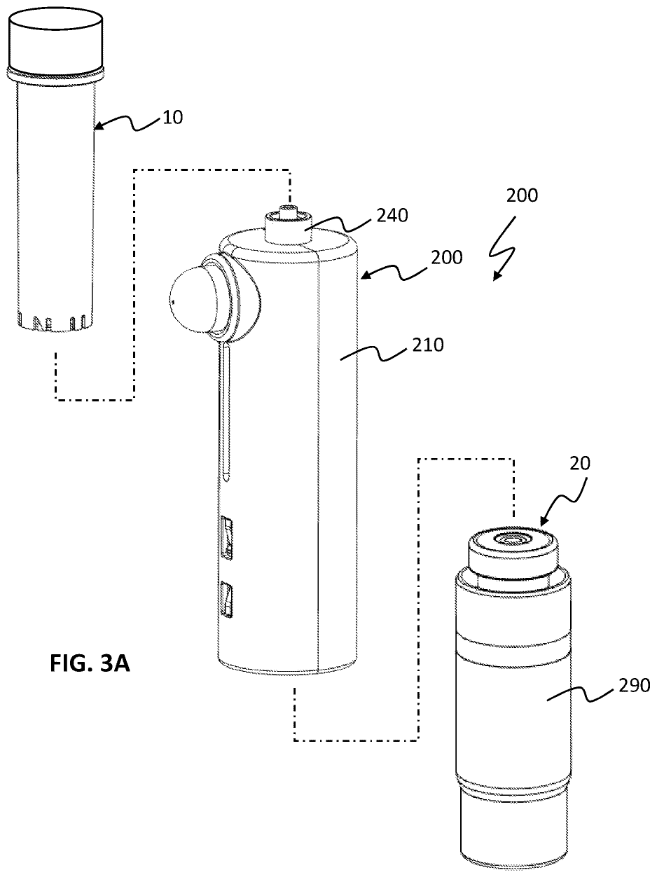

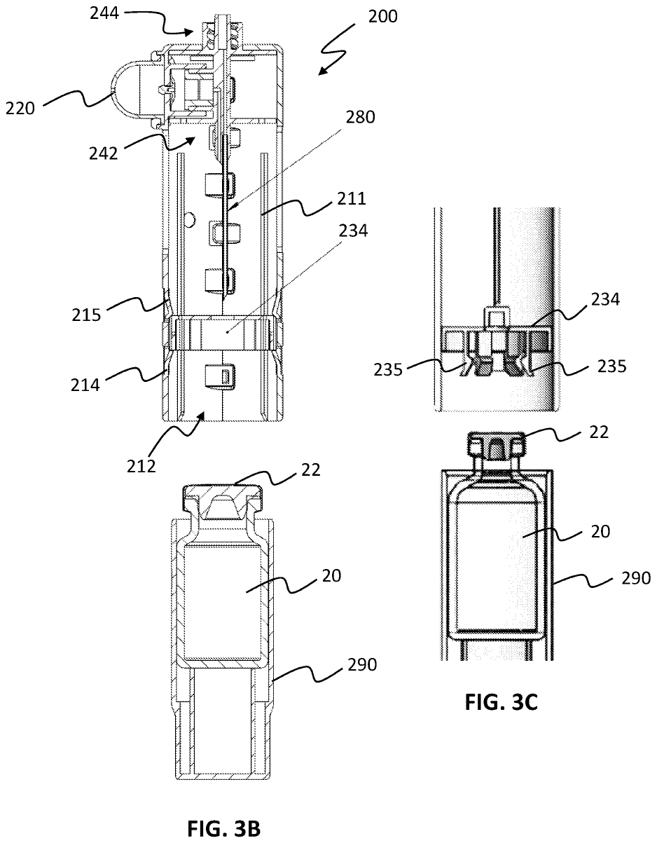

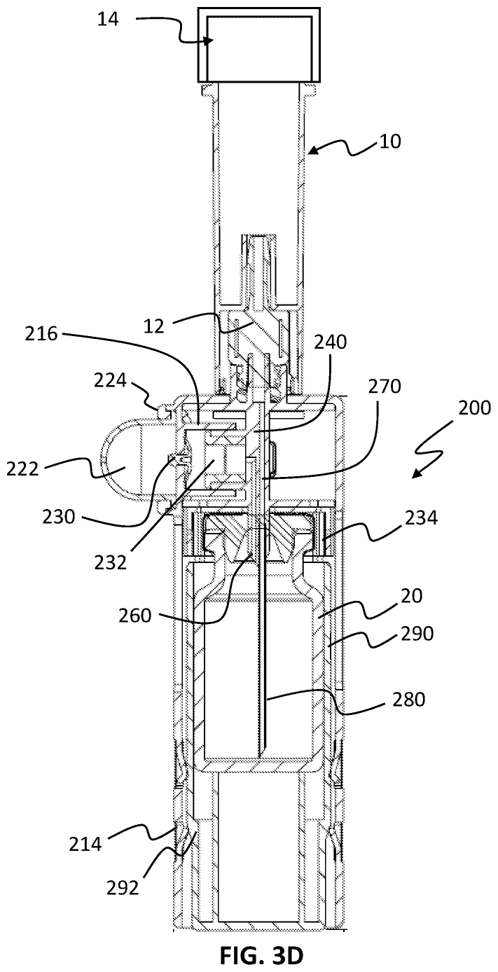

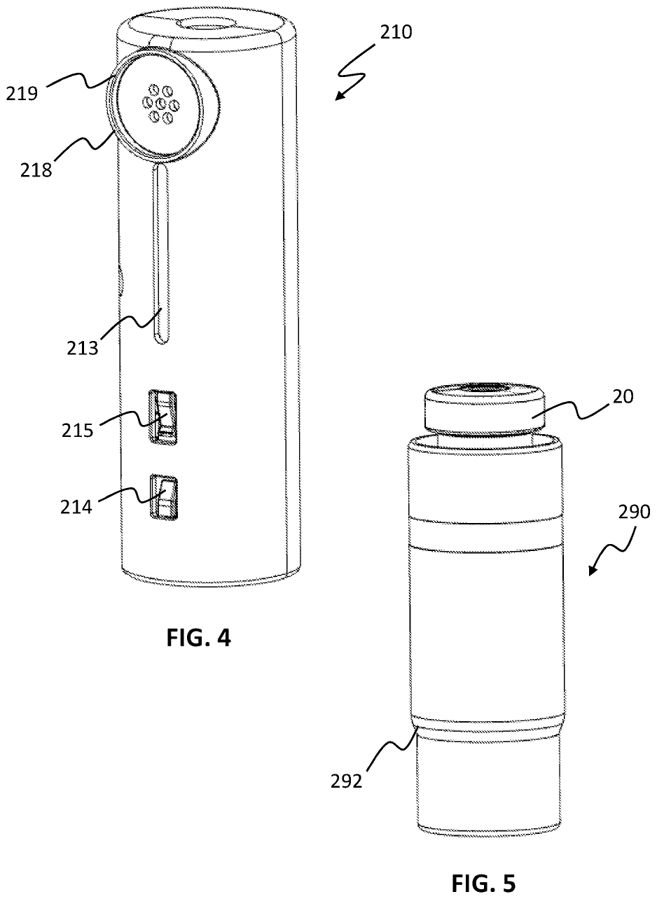

As shown in FIG. 4, the housing 210 may further include a pump setting 219 that receives the pump 220. The pump setting 219 may, for example, include a cradle to which the pump may couple. The pump setting 219 also may include one or more vents (e.g., holes) through which air or another suitable fluid may pass. As shown in FIG. 3D, in some variations, a one-way valve (e.g., umbrella valve 230) may open and permit airflow only in one direction through the vents, while substantially preventing airflow in the opposite direction through the vents. Specifically, when the pump 220 is not actuated, the umbrella valve 230 may be in a closed position such as that shown in FIG. 3D, substantially preventing airflow moving radially outward through the vents. When the pump 220 is actuated, the increased pressure may cause urge airflow radially inward through the vents, thereby causing the umbrella valve 230 to open by moving inwards into the housing 210. Accordingly, the valve may allow airflow into the housing, but substantially prevent airflow out of the housing. The airflow into the housing may then pass through at least one filter 232 that may, for example, be configured to remove particulates, microbes, moisture, etc. The filter may be a microbial barrier for the air that is pushed into the vial to force the liquid out of the vial and into the connected bottle, so that the air that contacts the interior of the vial is sterile. The filter may be of appropriate material and appropriate pore size suitable for filtering out microbes. The pore size may be for example between 0.22 microns and 0.2 microns. Some example materials for the filter may be be Nylon, PVDF-(Polyvinylidene fluoride), polypropylene, or PES (polyether sulfone). As shown in FIGS. 2B and 3D, the filter 232 may be seated in the adapter 240 or in any suitable location in the housing 210.

Pump 220 is shown in the figures as including a bulb 222 that couples to the pump setting 219 via a bulb ring 224 (e.g., that forms an air seal around the perimeter of the bulb 222). However, in other variations the pump 222 may be coupled to the pump setting 219 in any suitable manner, such as with an epoxy or other suitable gasket. Furthermore, the pump 220 may be constructed in any suitable manner. For example, instead of bulb 222, the pump 220 may include a button, slider, piston, crank, and/or the like. The pump 220 may be located on one side of the housing, or in any suitable location (e.g., a piston or other suitable pump that is axially oriented to the housing may similarly actuate airflow into the housing). The pump 222 may be manually actuated or automatically actuated (e.g., via an external interface). Although the pump 220 is shown as projecting laterally from the housing 210, it should be understood that the pump 220 may be positioned and/or constructed in any suitable manner (e.g., oriented longitudinally relative to the housing 210). In variations in which the pump 220 includes a depressible bulb 222, a depressed bulb may be refilled via a pin hole opening in the bulb.

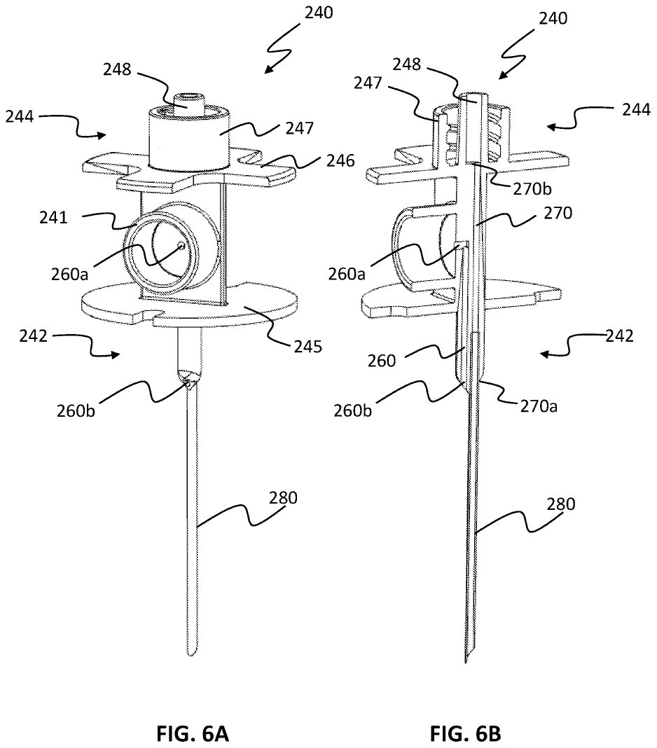

As shown in FIG. 2B, the drug transfer system 200 may further include an adapter 240. The adapter 240 may be arranged within the housing 210, and include at least a vial interface portion and a drug delivery device interface portion. For example, as shown in further detail in FIGS. 6A and 6B, the adapter 240 may include a vial interface portion 242 configured to be inserted into or otherwise engaged with a vial, and a drug delivery device interface portion 244 configured to engage a drug delivery device. Both the vial interface portion 242 and the drug delivery device interface portion 244 may be generally arranged along a central stem portion of the adapter 240. One end of the stem in the vial interface portion 242 is insertable into a vial, and the opposite end of the stem in the drug delivery device interface portion 244 may be coupleable to a drug delivery device.

In some variations, the adapter 240 may further include a filter setting 241 configured to receive a filter 232 as described above. The filter setting 241 may, for example, be located along the stem between the vial interface portion 242 and the drug delivery device interface portion 244. As shown in FIG. 3D, the filter setting 241 may be configured to mate with (e.g., align with) an adapter engagement setting 216 in the housing that circumscribes the filter setting 241, or coupled to the filter setting 241 in any suitable manner.

The first channel 260 and the second channel 270 function to provide ingress into the vial and egress out of the vial, respectively, when the vial interface portion 242 is engaged with a vial. As shown in FIGS. 6A and 6B, an inlet 260a of the first channel 260 may be located at the base of the filter setting 241. The first channel 260 may be defined in the stem of the adapter 240 as extending longitudinally along the stem from the inlet 260a to an outlet 260b. As shown in FIG. 6B, the second channel 270 may also be defined in the step of the adapter 240 as extending longitudinally along the stem (substantially parallel to the first channel 260) from an inlet 270a in the vial interface portion 242 to an outlet 270b in the drug delivery device interface portion 244.

The cannula 280 may be coupled to the second channel 270 (or otherwise in fluidic communication with the second channel), such as be being inserted into the second channel 270 with a mechanical interfit (e.g., press fit), epoxy, and/or other suitable technique. The cannula 280 may include sharpened distal tip, which may be configured to pierce a septum in the neck of a vial and help lead at least a portion of the vial interface portion 242 to be inserted in the vial. The cannula 280 may, for example, have a beveled tip. In some variations the cannula 280 may have a length that is about equal to the height or depth of a vial into which it will be inserted, so as to enable access to substantially all of the contents of the vial as the vial is emptied. However, the cannula 280 may alternatively have any suitable length.

In some variations, the adapter 240 may include a stop 245 that functions to provide an abutting surface against which the sharp guard 234 (described in further detail below) may stop when engaging a vial. The stop 245 may, for example, include a radially extending shoulder surface as shown in FIG. 6A. Additionally or alternatively, the stop 245 may include one or more latching arms (e.g., biased radially inward) with a lateral surface configured to engage the sharp guard, or any suitable stop feature. Additionally or alternatively, an internal surface of the housing 210 may include a stop surface that functions similarly to provide a surface to engage the sharp guard 234.

The drug delivery device interface portion 244 functions to engage a drug delivery device. For example, as shown in FIG. 6A, the drug delivery device interface portion 244 may include a luer engagement 247 configured to couple to a luer fitting of a drug delivery device. At least a portion of the luer engagement 247 may extend outside of the housing 210 and be exposed for coupling to the drug delivery device. The luer engagement 247 may be configured to removably couple to the luer fitting of the drug delivery device. For example, as shown in FIG. 6B, the luer engagement 247 may include threads configured to mate with a threaded connector on the luer fitting. The luer engagement 247 may further include a lumen 248 that is in fluidic communication with the outlet 270b of the second channel 270, and may engage the luer fitting in the drug delivery device. The luer engagement 247 may additionally or alternatively include any suitable engagement interface, such as one or more snap fit features (e.g., latches, etc.).

In some variations, the adapter 240 may include one or more alignment arms 246. The alignment arms 246 may, for example, function to axially and/or radially orient the adapter 240 within the housing 210. In some variations, the alignment arms 246 may extend radially from the central stem portion of the adapter 240, and include a lateral surface that abuts against a wall of the housing 210 to axially position the adapter 240 in a predetermined longitudinal position (e.g., to position the drug delivery device interface portion 244 at a particular position to ensure sufficient engagement with the drug delivery device). Additionally or alternatively, the alignment arms 246 may engage one or more radial features of the housing 210 to radially orient the adapter 240 into a predetermined rotational orientation (e.g., to rotationally align the filter setting 241 with the pump, filter, etc. as described above). Although four alignment arms 246 are shown in the figures, it should be understood that any suitable number of alignment arms (one, two, three, etc.) may be included in the adapter 240. Additionally or alternatively, the adapter and/or housing may include other suitable longitudinal and/or rotational alignment features for helping to seat the adapter in a predetermined position and/or orientation.

As shown in FIGS. 2B and 3B-3D, the drug transfer device 200 may further include a sharp guard 234. The sharp guard 234 may include a cover (e.g., radially extending surface) with a hole that enables the sharp guard 234 to be placed around the cannula 280. The sharp guard 234 may have a distal protective configuration at a distal portion of the cannula 280 so as to cover the sharp distal end of the cannula 280, thereby helping to prevent inadvertent injury from puncture by the cannula 280. The sharp guard 234 may be set in this distal protective configuration by being locked between radially projected features of the surrounding housing 210, such as between first and second radial spring arms 214 and 215 that are biased radially inward as shown in FIG. 3B. As another example, the sharp guard 234 may be biased toward this distal position with a biasing compressive spring (not shown) arranged around the cannula. A sufficient axial force (e.g., above a threshold force) may displace the sharp guard 234 from this distal protective configuration, thereby exposing the cannula 280.

The sharp guard 234 may be translatable along the cannula 280, such that it can be pushed toward the proximal end of the cannula 280. For example, the sharp guard 234 may travel axially within longitudinal guides 211 (shown in FIG. 3B) in the housing 210, until the sharp guard 234 reaches a proximal exposing configuration as shown in FIG. 3D. In this proximal position, the sharp guard 234 may, for example, engage the stop 245 of the adapter 240, as described above. In some variations, the adapter 240 and/or the internal surface of the housing 210 may also include one or more locking features (not shown) to help secure the sharp guard 234 in this proximal position, such as spring arm and/or the like that engages the sharp guard 234.

In some variations, the sharp guard 234 may also include one or more suitable feature(s) configured to couple to the vial 20. For example, as shown in FIG. 3C, the sharp guard 234 may include one or more vial engagement members 245 that are distributed around the sharp guard 234 in a radial configuration. The vial engagement members 245 may form a radial cradle configured to grasp and/or couple to a portion of the vial 20, such as around a neck or septum 22 of the vial 20. This coupling may include a snap fit interface, which may, for example provide tactile feedback to a user when a vial has been inserted in the housing 210 and is coupled to the sharp guard 234.

The drug transfer system may further include a carrier 290 that functions to help convey the vial 20 into the vial receptacle. For example, as shown in FIGS. 3A-3C, the carrier 290 may include a cavity or recess that receives the vial 20, and the carrier 290 may be insertable into the vial receptacle. The carrier 290 is shown in the figures as generally cylindrical or having a circular cross-sectional shape, for receiving a cylindrical vial. However, the carrier 290 may have any suitable shape (e.g., prismatic with any polygonal cross-section, elliptical cross-section, tapered, etc.). Furthermore, the vial 20 may be axially and/or rotationally fixed or locked in the carrier 290 with a suitable locking feature (e.g., latch, detents, frictional textured surface(s), and/or conformable material(s) on an external surface of the vial 20 and/or internal surface of the carrier 290, etc.).

In some variations, carrier 290 may be sized and shaped such that at least a portion of the vial 20 (e.g., the neck portion and/or septum 22 of the vial) remain outside of the carrier 290, so as to help facilitate the engagement between the sharp guard 234 and the vial 20. For example, the depth of the cavity in the carrier may be less than the overall height of the vial 20. Alternatively, the cavity may vary in diameter (e.g., having a narrower diameter at a base of the cavity relative to the opening of the cavity), though a portion of the cavity may have a diameter or width that is at least as wide as the vial 20 (and thus accommodates the vial 20), and the depth of that portion of the cavity may be less than the overall height of the vial 20, such that at least a portion of the vial 20 is not within the cavity. However, in some variations the cavity is sized and shaped such that the entire vial 20 is recessed within the cavity (e.g., the depth of the cavity is greater than the height of the vial 20).

The carrier 290 and/or the housing 210 may include one or more engagement features to help retain the carrier 29 within the vial receptacle 212. For example, as shown in FIG. 5, the carrier 290 may include a shoulder 292 having a sloped or otherwise angled surface. The shoulder 292 may be configured to engage with a spring arm (e.g., spring arm 214 or spring arm 215) of the housing 210 when the carrier 290 is inserted into the housing 210 at a certain threshold insertion depth. For example, as shown in FIG. 3D illustrating the carrier 290 fully inserted in the vial receptacle, the spring arm 214 may be configured to radially engage the shoulder 292, thereby functioning as a latch that substantially prevents movement of the carrier 292 out of the housing 210, thus securing the vial 20 within the carrier 290 and housing 210. However, the carrier 290 and/or housing 210 may additionally or alternatively include any other suitable engagement features. For example, in some variations the carrier 290 may include external threads configured to mate with internal threads of the vial receptacle 212, such that removal of the carrier 290 requires substantial intention to rotate the carrier relative to the housing sufficiently to unscrew the carrier 290 from the vial receptacle 212. Alternatively, in this variation the threaded interface between the carrier 290 and the vial receptacle may include self-locking threads to substantially prevent unscrewing motion that would permit removal of the carrier 290 from the vial receptacle 212.

In other variations, the carrier 290 may be omitted from the drug transfer system, and the vial 20 may be inserted directly into the vial receptacle 212. In these variations, the vial 20 and/or the vial receptacle 212 may include one or more engagement features to help retain the vial 20 within the vial receptacle 212. For example, the vial 20 may include a shoulder surface similar to shoulder 292 described above in the carrier 290, where the vial's shoulder surface may engage with a spring arm 214 or spring arm 215 in the housing 210 to lock the vial 20 in the vial receptacle 212. Additionally or alternatively, the vial 20 may be have other suitable engagement features in the vial body, vial neck, vial septum, etc. (e.g., a threaded vial body).

FIGS. 3A-3D generally illustrate an example method of using the drug transfer system 200 to aseptically transfer liquid drug contents of a vial 20 to a drug delivery system 10. As shown in FIG. 3A, a drug delivery device 10 may be coupled to one end of the drug transfer system 200, such as via the adapter 240 extending from the housing 210. As shown in FIGS. 3A and 3B, a vial 20 (including contents to be transferred) may be placed in a carrier 290, and the carrier 290 may be inserted into the vial receptacle in the housing 210, on an end of the housing 210 opposite the adapter 240. As the carrier 290 is inserted deeper into the housing 210, the vial 20 and/or the carrier 290 displace the sharp guard 234 toward a proximal end of the cannula 280.

During this process, the first channel 260 and the cannula 280 (and/or second channel 270) may be allowed to extend into the vial. The sharpened distal tip of the cannula 280 and/or second channel 270 may pierce any septum that is present at the top of the vial 20 as the carrier 290 is inserted into the housing 210.

At a certain insertion depth (e.g., when the sharp guard 234 hits the stop 245 on the adapter 240), further insertion of the carrier 290 may cause the carrier 290 to couple to the sharp guard 234. Specifically, as shown in FIG. 3D, at this certain insertion depth (a seated “home” position for the carrier 290), further insertion of the carrier 290 may cause the engagement members 235 on the sharp guard 234 to surround and grasp the neck of the vial 20, thereby coupling the vial 20 to the sharp guard 234. Once the vial 20 is coupled to the sharp guard 234, the vial 20 may be locked in the housing 210 and substantially prevented from being removed from the housing 210 in the reverse direction. Additionally or alternatively, when the carrier 290 is at the home position as shown in FIG. 3D, the spring arm 214 may radially engage the shoulder 292 of the carrier, also helping to prevent removal of the carrier 290 and the vial 20 from the housing 210.

Once the carrier 290 and vial 20 are secured in the home position shown in FIG. 3D, a user may actuate the pump 220 (e.g., by repeatedly pressing the bulb 222). This drives air into the first channel 260 and into the vial 20, thereby urging the liquid drug contents of the vial 20 through the cannula 280 (if present), through the second channel 270, and into the drug delivery device 10. This transfer of drug from the vial 20 to the sterilized drug delivery device 10 is within a closed, aseptic environment, and is thus an aseptic transfer process.

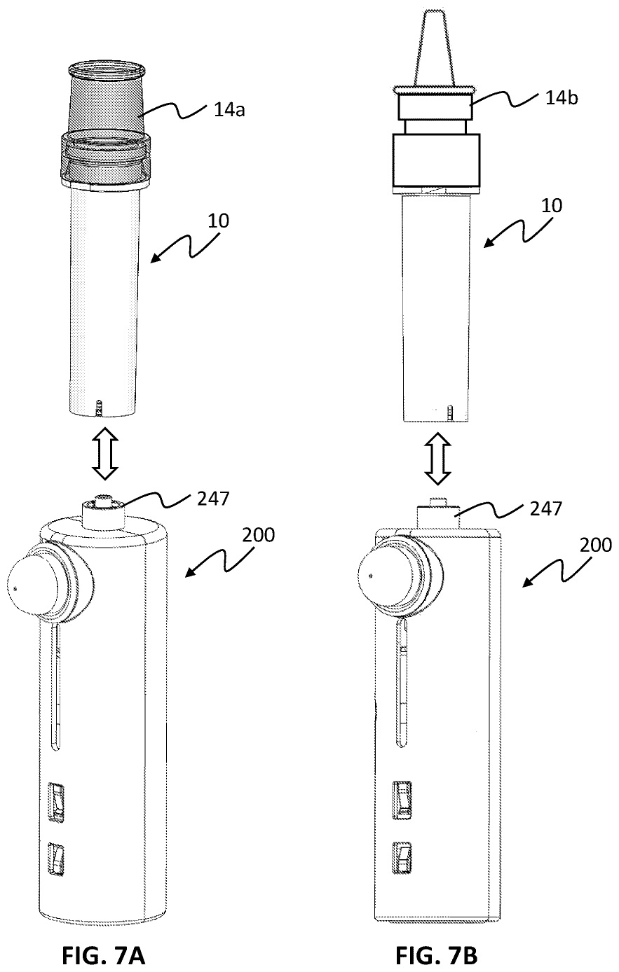

Once a desired volume of liquid has been transferred, the drug delivery device 10 may be decoupled from the drug transfer system 200. The detached drug transfer device 200 may then be discarded, or reused (e.g., after a sterilization process) for another drug transfer process. The detached drug delivery device 10 may be operated to administer the drug in accordance with the dispenser 14 attached to the drug delivery device 10. For example, FIG. 7A illustrates an example in which the drug transfer system 200 is operated with a drug delivery 10 including an eye dropper dispenser 14a. As another example, FIG. 7B illustrates an example in which the drug transfer system 200 is operated with a nasal spray pump 14b.

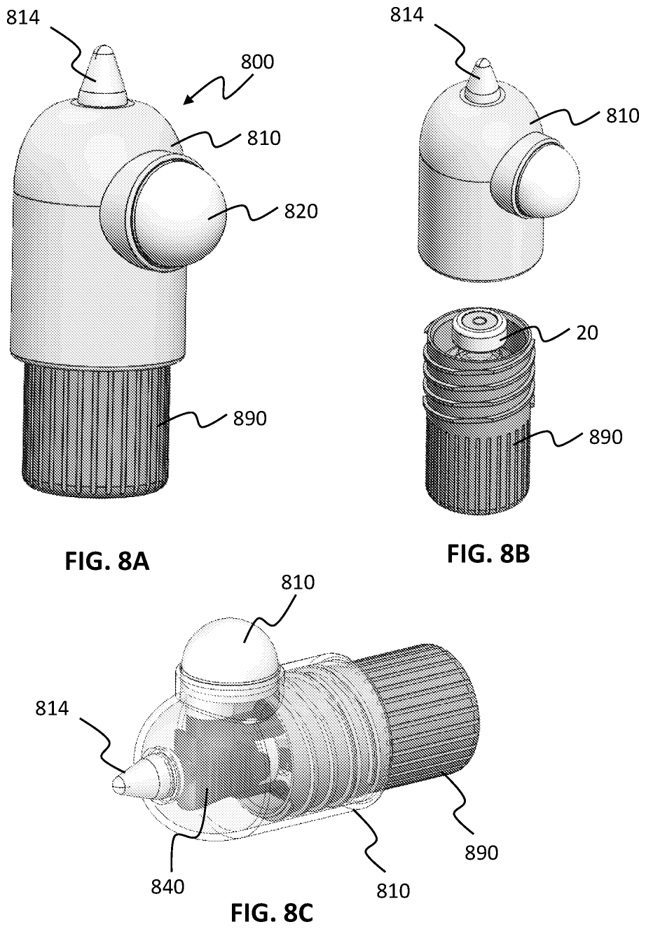

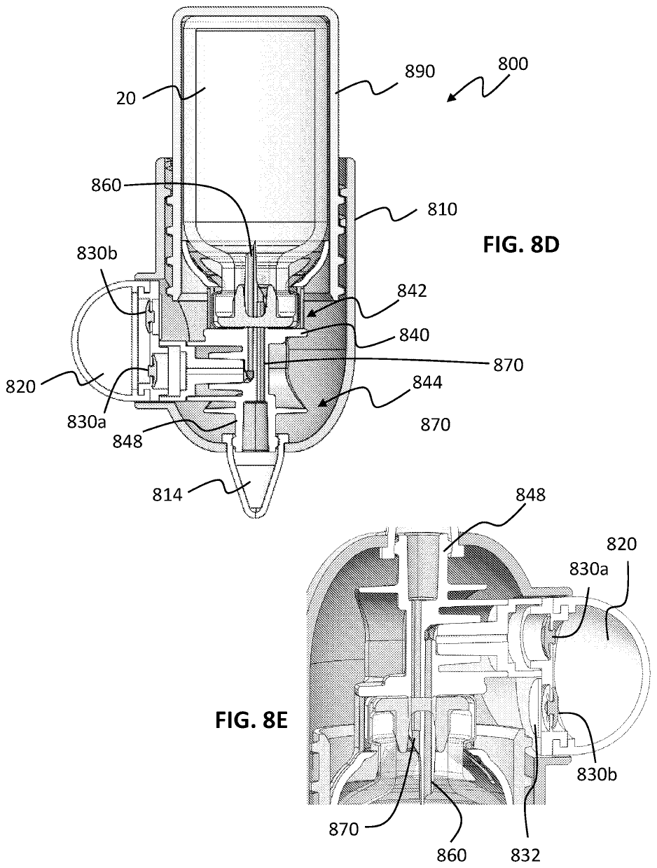

Described above are example variations of drug transfer systems in which a separate drug delivery device is removably coupled to the drug transfer systems. However, in other variations, the drug transfer system may include an integrated drug transfer system. For example, FIGS. 8A-8E illustrate an example variation of a drug transfer system 800 including a housing 810, a carrier 890 configured to engage the housing 810 to form an enclosed vial receptacle, and a drug dispenser 814 in fluidic communication with the vial receptacle. The drug transfer system 800 may further include a pump 820 that is configured to pressurize a vial 20 placed in the vial receptacle and drive liquid drug contents from the vial 20 in a manner similar to that described above with respect to drug transfer system 200, except that the drug transferred from the vial 20 may be passed directly to the integrated drug dispenser 814.

As shown in FIG. 8B, the carrier 890 may be configured to engage the housing 810 so as to collectively form an enclosed space functioning as a vial receptacle. The carrier 890 may, for example, include a hollow cavity for receiving the vial 20, and the carrier 890 may include a threaded interface that mates with a corresponding threaded interface of the housing 810 to close an open end of the hollow cavity. As such, when the carrier 890 and the housing 810 are coupled as shown in FIGS. 8A and 8C, a vial placed inside the carrier 890 is enclosed. The coupling of the carrier 890 and the housing 810 may be removably coupled or substantially permanently coupled (e.g., with self-locking threads). Furthermore, although the carrier 890 and the housing 810 are shown as being coupleable via a threaded interface, it should be understood that the carrier 890 and the housing 810 may couple in any suitable manner (e.g., one or more fasteners, an integrated latch or clip, a snap fit interface with interlocking engagement features, and/or other suitable mechanical interfit, etc.).

The pump 820 may be similar to any of the pumps in the drug transfer systems described above (e.g., pump 220). For example, the pump 820 may include a manually actuation element such as a bulb (as shown in FIG. 8A), button, slider, piston, trigger, lever, and/or the like.

Although the pump 820 is shown as projecting laterally from the housing 810, it should be understood that the pump 820 may be positioned and/or constructed in any suitable manner (e.g., oriented longitudinally relative to the housing 810).

In some variations, an adapter 840 may be arranged in the housing 810, and may include one or more fluidic channels. For example, the adapter 840 may define one or more channels that are functionally similar to the first channel (e.g., first channel 160, first channel 170), the second channel (e.g., second channel 260, 270), and/or the cannula (e.g., cannula 280) described above. As shown in FIG. 8D, for example, the adapter 840 may include a vial interface portion 842 and a drug dispenser interface portion 844. A first channel 860 may be in fluidic communication with the pump 820 and extend at least partially into the vial receptacle. The first channel 860 may include a sharpened or beveled distal tip, such as for piercing a septum of the vial 20 placed in the vial receptacle. In some variations, the housing 810 and/or the carrier 890 are sized such that in the process of coupling the housing 810 and carrier 890 together with a vial 20 placed therein, the first channel 860 is urged toward the vial 20 and pierces a septum of the vial 20 to permit fluidic access to the liquid drug contents of the vial 20. The second channel 870 may be in fluidic communication with the vial receptacle such that is at least partially inserted in the vial 20 when the vial is placed in the vial receptacle.

In operation of the drug transfer system 800 fully assembled with a vial 20 as shown in FIG. 8D, actuation of the pump 820 may induce air (or other suitable fluid) into the first channel 860 and into the vial 820. Specifically, actuation of the pump 820 (e.g., depressing the bulb of pump 820) may force air through a first one-way valve (e.g., umbrella valve 830a shown in FIG. 8E) that is arranged between the pump 820 and the first channel 860. The air may pass through the valve and through the first channel 860 that is extended into the vial 20, thereby pressurizing the interior of the vial 20. The increased pressure within the vial 20 urges the liquid drug contents of the vial 20 into the second channel 870, transferring the drug to the drug dispenser 814. The transferred drug may then be administered by the drug dispenser 814 in accordance with the dispenser type. For example, the dispenser 814 shown in the figures includes a nozzle tip suitable as an eyedropper. As such, in this variation, the drug transfer system 800 may be inverted such that the eyedropper dispenser 814 is oriented downward (e.g., toward an eye), such that when the pump 820 is actuated, the eyedropper dispenser 814 may immediately administer the drug that has been transferred from the vial 20 by the drug transfer system 800.

As described above, depressing the bulb of the pump 820 may cause a volume of the liquid drug to be transferred from the vial 20 to the dispenser 814. In some variations, the pump 820 may be sized to induce the transfer of a desired, predetermined volume of the liquid drug from the vial. This predetermined volume may, for example, correspond to (e.g., equal to a single dose of the drug, or a desired fraction of the dose such as one drop of a prescribed two-drop dose). For example, the size of the bulb for the pump 820 may be selected based on a desired volume to be transferred for each depression of the bulb.

In some variations, when the pump 820 is released (e.g., the bulb is released), the first one-way valve (e.g., umbrella valve 830a) may close, and the pressure change may allow a second one-way valve (e.g., umbrella valve 830b) to open. The opening of the second valve may allow air to flow through a filter 832 (e.g., similar to filter 232 described above) and through the opened second valve to refill the bulb of the pump 820. Once the bulb is sufficiently refilled and pressurized, the second valve may re-close, returning the bulb to a neutral state ready to be depressed again if a user desires to administer more drug from the dispenser 814.

The drug transfer system 800 is described above as including two valves managing air communication with the bulb of the pump 820. However, a similar variation of a drug transfer system 800 may instead include only one valve for managing such air communication, with a filter (similar to filter 832) that is placed between the valve and the first channel 860. In this variation, instead of being refilled via a separate path through a second valve as described above, the bulb may be refilled via a pin hole opening in the bulb.

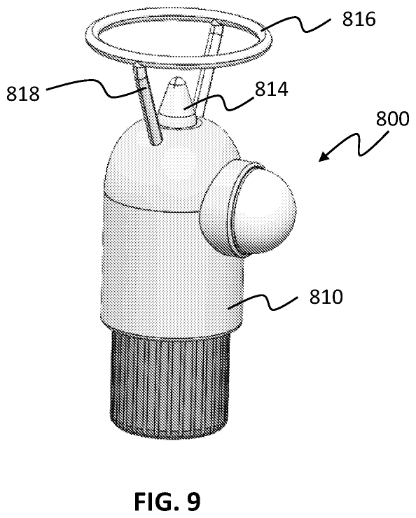

As shown in FIG. 9, an example variation of a drug transfer system 800 may include an eyedropper dispenser, where the drug transfer system 800 further includes a guard 816 configured to help prevent the eyedropper dispenser 814 from contacting the eye when the drug transfer system 800 is in use. The guard 816 may, for example, have an enclosed shape (e.g., circular or elliptical ring) sized and shaped to be placed in contact with a user's face around the eye (e.g., around the eye socket, or orbit). The guard 816 may furthermore be contoured, such as in a saddle or cup shape, to improve conformity against the user's face and/or to improve comfort. Comfort may be further improved with padding and/or the like. The guard 816 may, in some variations include one or more anti-infective materials (e.g., anti-microbial, anti-fungal, etc.), such as silver-impregnated materials, to help limit bacterial and other growth on the guard 816.

The user-contacting side of the guide 816 may be spaced apart from the distal tip of the eyedropper dispenser 814. Specifically, the guard 816 may be offset from the dispenser 814 (or from the housing 810) by a distance sufficient to enforce a buffer space between the dispenser 814 and the user's eye, when the guard 816 is placed around the user's eye. This offset distance may be defined at least in part by the length of guard mount(s) 818 (e.g., arms or other suitable members), which in some variations may be angled radially outward to help improve stability of the guard 816. In some variations, a plurality of such guard mounts may be distributed around the perimeter of the guard 816. Furthermore, although separate, distinct guard mounts are shown in FIG. 9, it should be understood that the guide 816 may be coupled to and/or offset from the housing 810 in any suitable manner (e.g., a cup, skirt, etc.).

Although the drug transfer system 800 is shown and described above with respect to FIGS. 8A-8E as including an eyedropper dispenser, other variations of a drug transfer system may include any suitable type of dispenser. For example, in some variations the dispenser 814 may include a nasal spray pump.

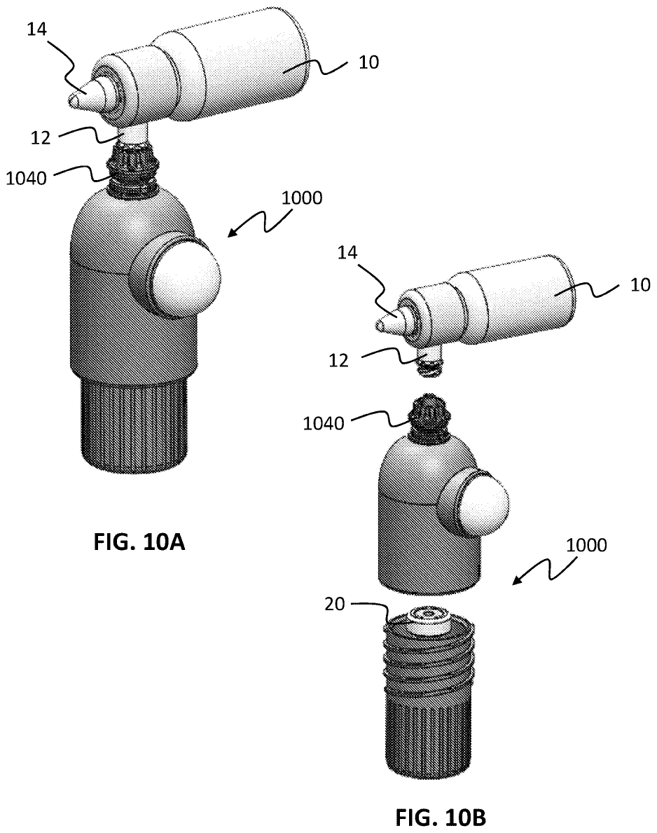

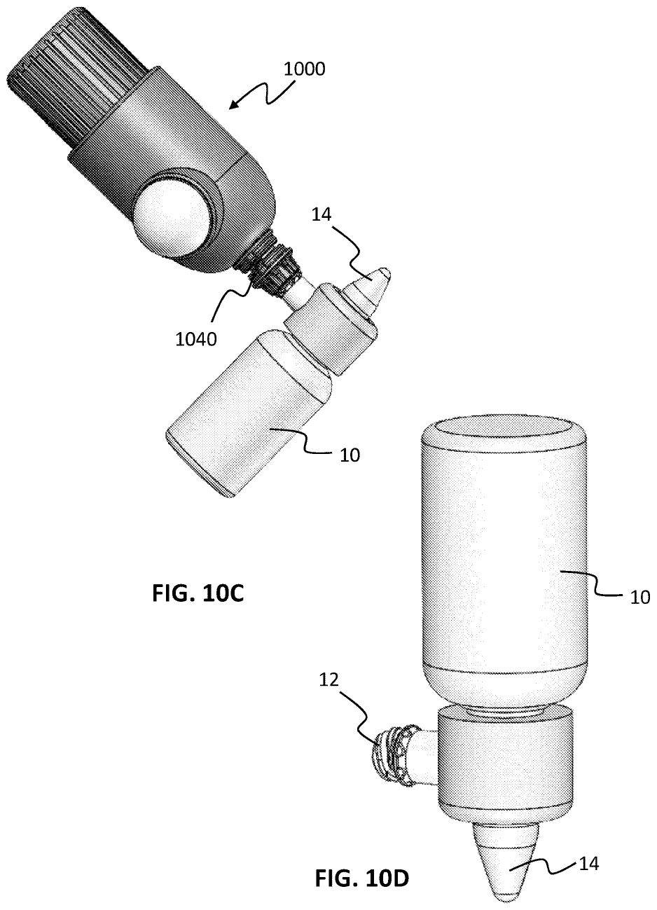

It should be understood that various features of the multiple variations of drug transfer systems may be combined in any suitable manner. For example, FIGS. 10A-10C illustrate an example variation of a drug transfer system 1000 that is similar to the drug transfer system 800 described above with respect to FIGS. 8A-8E, except with respect to the drug dispenser. As shown in FIGS. 10A-10C, the drug transfer system 1000 may include an adapter 1040 configured to couple (e.g., removably couple) to a separate drug delivery system in a manner similar to that described above with respect to drug transfer system 200. For example, the adapter 1040 may be configured with a suitable luer engagement feature that is configured to couple to a luer fitting 12 of the drug delivery device 10. Liquid drug contents of a vial 20 may be aseptically transferred from the vial 20 as described above with respect to the drug transfer system 800, then driven through the adapter 1040, through the luer fitting 12, and into the drug delivery device 10. After a desired amount of drug has been transferred to the drug delivery device 10, the drug delivery device 10 may be decoupled from the drug transfer device 1000 (FIG. 10D) and be operated to administer the drug as desired. The detached drug transfer device 1000 may then be discarded, or reused (e.g., after a sterilization process) for another drug transfer process.

Described above are example variations of drug transfer systems in which the drug transfer system incorporates an integrated pump. However, in other variations, the drug transfer system may lack an integrated pump and may be configured so that when a drug delivery device (optionally with a dispenser) is coupled to the drug transfer system, the drug delivery device functions as the pump that is configured to pressurize a vial 20 placed in the vial receptacle and drive liquid drug contents from the vial 20 through the drug transfer system to the drug delivery device in a manner similar to that described above with respect to drug transfer system 100, 200, 800 and the like. In such a configuration, the drug delivery device when may be considered to be part of the drug transfer system.

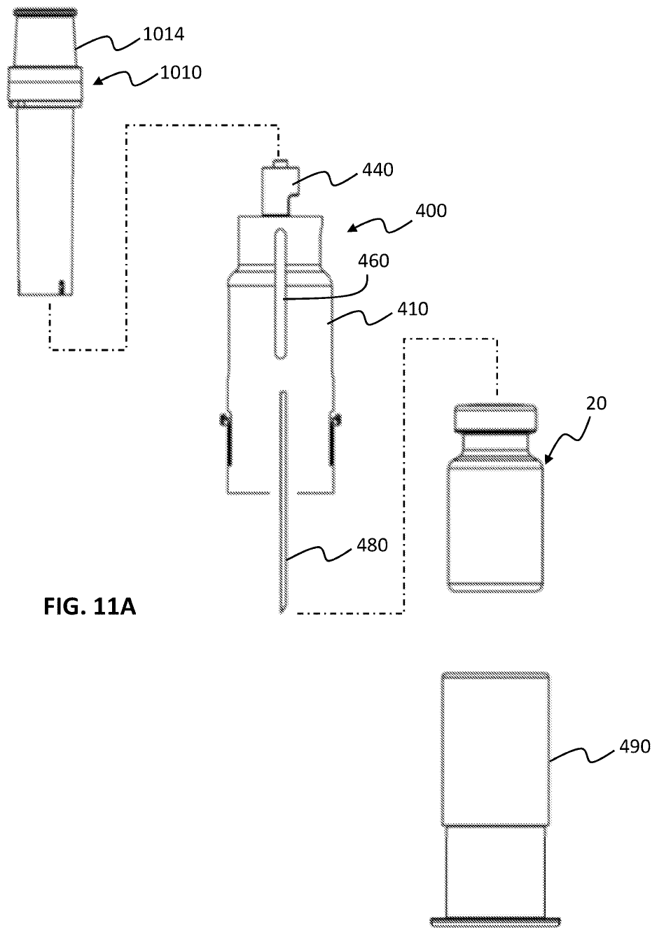

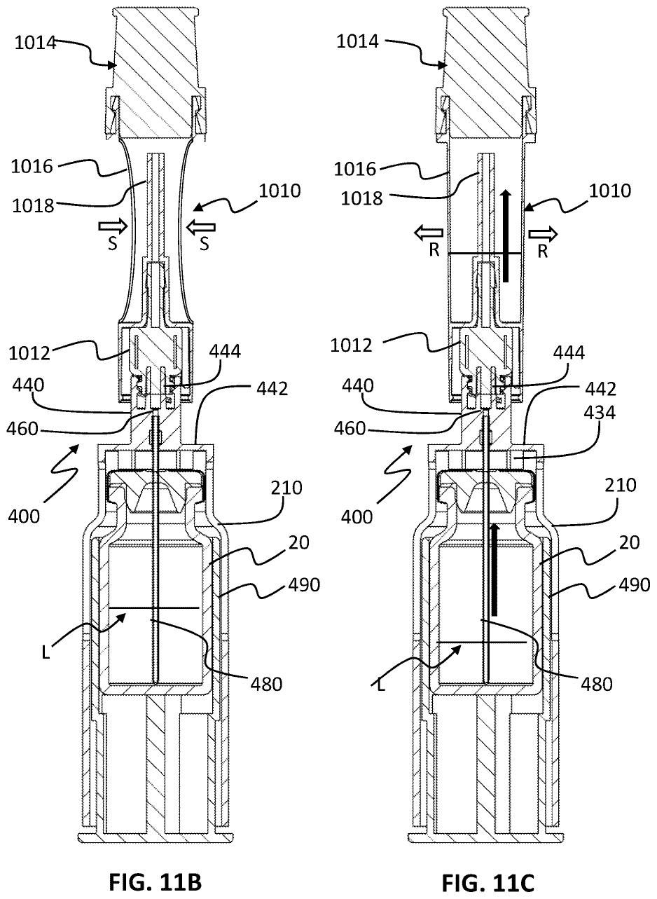

For example, FIGS. 11A-11C illustrate an example variation of a drug transfer system 400 including a housing 410, a vial carrier 490 configured to engage the housing 410 to form an enclosed vial receptacle configured to hold a vial 20 and a drug delivery device 1010, optionally comprising drug dispenser 1014, in fluidic communication with each other.

As shown in FIG. 11A, the carrier 490 may be configured to engage the housing 410 so as to collectively form an enclosed space functioning as a vial receptacle configured to securely hold a vial 20. The carrier 490 may, for example, include a hollow cavity for receiving the vial 20, and the carrier 490 may include a threaded interface that mates with a corresponding threaded interface of the housing 410 to close an open end of the hollow cavity. As such, when the carrier 490 and the housing 410 are coupled as shown in FIGS. 11B and 11C, a vial 20 placed inside the carrier 490 is enclosed. The coupling of the carrier 490 and the housing 410 may be removably coupled or substantially permanently coupled (e.g., with self-locking threads). It should be understood that the carrier 490 and the housing 410 may couple in any suitable manner (e.g., one or more fasteners, an integrated latch or clip, a snap fit interface with interlocking engagement features, and/or other suitable mechanical interfit, etc.).

As shown in the schematic of FIG. 11A-11C, in some variations, the drug transfer system 400 may include a cannula 480 configured to extend at least partially into the vial receptacle. The drug transfer system 400 may also include a first fluidic channel 460 defined in an adapter 440. The first channel 460 may be in fluidic communication with the cannula 480 on one end, and be in fluidic communication with the drug delivery device 1010 when the drug delivery device is connected to the drug transfer system. When a vial 20 is inserted into the vial receptacle, the cannula 480 may enter and extend at least partially into the vial 20. In some variations, the cannula 480 may extend nearly the entire depth of the vial 20 so that the cannula terminates in the liquid drug contents of the vial 20, below level (L).

As shown in the schematic of FIG. 11A-11C, in some variations, the drug delivery device 1010 (with dispenser 1014) may be coupled to the drug transfer system 400 to receive a fluid from the vial 20. The drug delivery device 1010 may, for example, couple to the drug transfer system 400, which may comprise the adapter 440, so as to be in fluidic communication with the vial 20 through the first channel 460 and the cannula 480. The drug delivery device 1010 may comprise an internal channel 1018 in fluid communication with the first channel and the cannula. The internal channel 1018 if present raises the point of exit/entry in the interior of the drug delivery device 1010, and channel 1018 may thereby function to preserve liquid that may already be present within the drug delivery device and prevent the liquid from re-entering the first channel 460 and the cannula 480, and into vial 20.

The adapter 440 may include a vial interface portion 442 configured to be inserted into or otherwise engaged with a vial, and a drug delivery device interface portion 244 configured to engage the drug delivery device 1010, by way of example a luer fitting 1012 comprised in the drug delivery device 1010. The adapter 440 may be configured so that the vial 20 engages with the vial interface portion 442 and the drug delivery device 1010 engages with the device interface portion 244 in an airtight manner so that squeezing the walls 1016 of the drug delivery device drives air into the vial 20 through the first channel 160 and cannula 480. Both the vial interface portion 242 and the drug delivery device interface portion 244 may be generally arranged along a central stem portion of the adapter 240.

Generally, the drug transfer system 400 and drug delivery device 1010 may be formed through any suitable manufacturing process, such as injection molding, 3D printing, casting, and/or suitable machining processes. Suitable materials for forming any of the components of the drug transfer system 400 include rigid or semi-rigid plastics, though the drug transfer system 400 may include any suitable material. The walls of the drug delivery device 101 may be composed of a compressible semi-rigid material, such as a compressible semi-rigid plastic, that is configured to return to its original shape following compression, for example when a user squeezes the walls 1016.

To operate the drug transfer system 400, as shown in FIGS. 11B-11C, a user may squeeze the walls 1016 of the drug delivery device 1010 to operate the drug delivery device as a pump to drive air into the vial 20 and increase the pressure in the vial 20. See open block arrows S schematically showing the squeezing of walls 1016. As shown in FIG. 11C, release of the walls 1016, schematically shown as open block arrows R, causes the pressure within the drug delivery device 1010 to be reduced relative to the interior of the vial 20, thus driving the drug in the vial into the cannula 480, through the first channel 460, and into the drug delivery device 1010 (through the luer fitting 1012, if present). The flow of liquid from the vial 20 into the drug delivery device 1010 is shown schematically as filled block arrows FIG. 11C. Accordingly, squeezing followed by release of the wall 1016 of the drug delivery device 1010 may result in a transfer of the liquid product from the vial 20 into the drug delivery device 1010. It will be appreciated that the internal channel 1018, when present, would prevent liquid already drawn from the vial 20 into the drug delivery device from being returned into the vial when the walls are squeezed, while not interfering with the transfer of liquid from the vial into the drug delivery device.

After a sufficient amount of liquid product has been transferred, the drug delivery device 1010 may be detached from the drug transfer system 400. The luer fitting 1012 (if present) of the drug delivery device 1010 may be sealed (e.g., self-sealing septum, covered with a cap component, etc.). Once decoupled from the drug transfer system 400, the drug delivery device 1010 may be used separately to administer the drug to a patient in accordance with the particular dispenser in the drug delivery device (e.g., eyedropper, nasal spray pump).

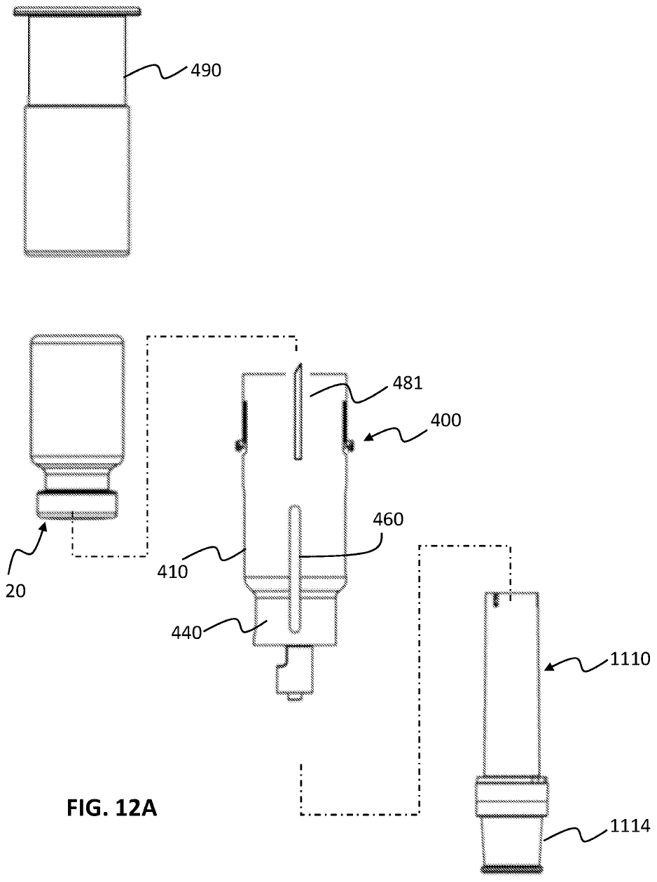

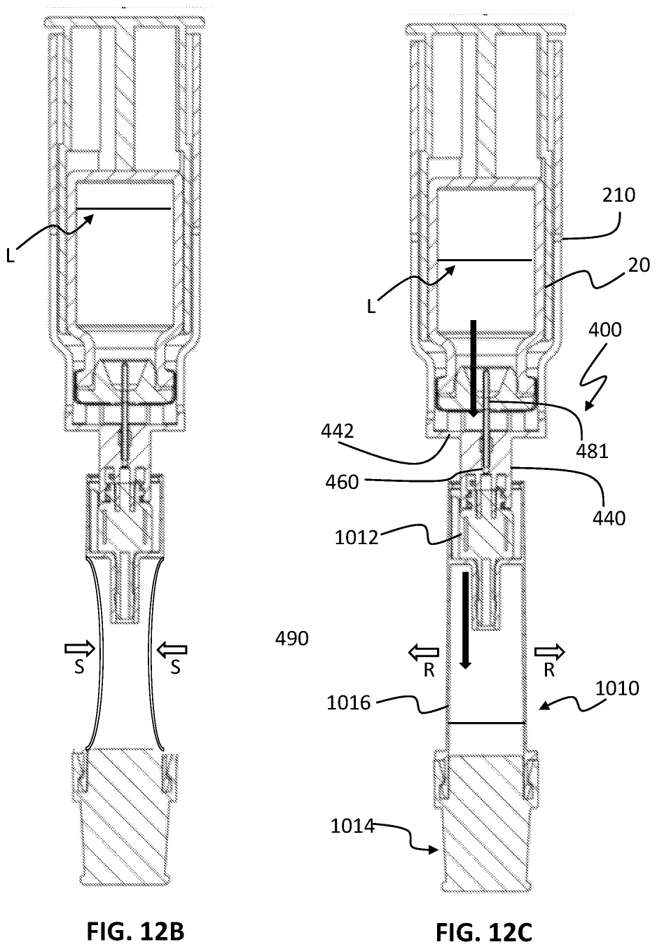

In the example drug transfer system 400 described with respect to FIGS. 11A-11C, the system is used in an “upright” configuration, with the vial 20 situated below the drug delivery device 1010. As shown in FIGS. 12A-12C, the system may alternatively be used in an “inverted” configuration, with the vial 20 situated above a modified drug delivery device 1110. In the inverted configuration, the cannula 481 that is inserted into the vial 20 may be short relative to the cannula 480 that is used in the upright configuration so that the cannula's tip is near the vial septum 22 and therefore terminates in the liquid drug contents of the vial 20, below level (L).

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A drug transfer system comprising:
a housing comprising a vial receptacle;
a pump coupled to the housing;
an adapter arranged in the housing and comprising:
a vial interface portion and a drug delivery device interface portion;
a first channel defined at least partially in the vial interface portion, wherein the first channel is in fluidic communication with the pump and the vial receptacle;
a second channel defined a least partially in the drug delivery device interface portion; and
a cannula in fluidic communication with the second channel and extending at least partially into the vial receptacle; and
wherein the drug transfer system is coupleable to a drug delivery device such that the drug delivery device is in fluidic communication with the second channel.

2. The system of claim 1, further comprising a sharp guard slidingly engaged with the cannula.

3. The system of claim 2, wherein the sharp guard comprises one or more members configured to engage a vial.

4. The system of claim 1, wherein the pump is configured to pressurize the vial receptacle.

5. The system of claim 4, wherein the pump is manually actuatable.

6. The system of claim 5, wherein the pump comprises at least one actuator selected from the group consisting of a bulb, a button, a trigger, a piston, a slider, and a crank.

7. The system of claim 1, further comprising a carrier insertable in the vial receptacle.

8. The system of claim 7, wherein the carrier comprises a cavity configured to receive a vial.

9. The system of claim 7, wherein at least one of the housing and the carrier comprises a lock configured to retain the carrier in the vial receptacle.

10. The system of claim 9, wherein the housing comprises a lock comprising a spring arm configured to engage a surface of the carrier.

11. The system of claim 1, further comprising a one-way valve in a fluidic communication path between the pump and the vial receptacle.

12. The system of claim 11, further comprising a filter in the fluidic communication path between the pump and the vial receptacle.

13. The system of claim 1, wherein the drug transfer system is removably coupleable to the drug delivery device.

14. The system of claim 13, wherein the drug transfer system is removably coupleable to the drug delivery device via the drug delivery device interface on the adapter.

15. The system of claim 14, wherein the adapter comprises a luer-compatible interface removably coupleable to the drug delivery device.

16. The system of claim 1, wherein the adapter comprises a threaded interface.

17. A method of aseptically transferring a drug to a drug delivery device, comprising:
coupling a drug delivery device to a drug transfer device comprising vial receptacle, a pump, a first channel in fluidic communication with the pump and the vial receptacle, and a second channel in fluidic communication with the drug delivery device and extending at least partially into the vial receptacle;
inserting, into the vial receptacle, a vial comprising the drug to be transferred;
allowing the first channel and the second channel to extend into the vial;
actuating the pump to drive fluid into the first channel and into the vial, thereby urging the drug through the second channel and into the drug delivery device; and
decoupling the drug delivery device from the drug transfer device.

18. The method of claim 17, further comprising coupling the vial to a carrier, wherein the carrier is insertable in the vial receptacle.

19. The method of claim 18, further comprising locking the carrier in the vial receptacle.

20. The method of claim 17, wherein the second channel comprises a cannula configured to extend into the vial.

21. A method of administering a drug to a patient, the method comprising the steps of:
coupling a drug delivery device to the drug transfer system of claim 1, wherein the drug is included within the vial receptacle of the drug transfer system;
aseptically transferring the drug from the drug transfer system to the drug transfer device; and
administering the drug to a patient.

22. The method of claim 21, wherein the drug delivery device comprises an eyedropper dispenser or a nasal spray pump dispenser.

23. The method of claim 21, further comprising the step of decoupling the drug delivery device from the drug transfer system prior to administering the drug to the patient.

24. The method claim 21, wherein the drug is administered to the patient prior to decoupling the drug delivery device from the drug transfer system.

25. The method of claim 24, wherein the drug transfer system is reused.

26. The method of claim 21, further comprising the step of actuating a pump in order to administer the drug to the patient.

27. The method of claim 26, wherein the administering of the drug constitutes a first administration, which is followed by at least one more administration of the drug.

28. The method of claim 21, wherein a user administers the drug.

29. The method of claim 28, wherein the user is the patient.
